(19) United States
(12) Patent Application Publication
(10) Pub. No.: US 2007/0298455 A1
(43) Pub. Date: Dec. 27, 2007

(54) Method For Preparing A Modified Host Cell

(76) Inventor: Marco Alexander Van Den Berg, Poeldijk (NL)

Correspondence Address: NIXON & VANDERHYE, PC, 901 NORTH GLEBE ROAD, 11TH FLOOR, ARLINGTON, VA, 22203, US

(21) Appl. No.: 10/575,369

(22) PCT Filed: Oct. 14, 2004
(86) PCT No.: PCT/EP04/11615

§ 371 Date: Apr. 11, 2006

(30) Foreign Application Priority Data
Oct. 14, 2003 (EP) 03078253.6

(51) Int. Cl. C12P 21/06; C07H 21/00; C12N 15/01; C12N 15/09; C12P 33/00; C12P 1/00; C12P 19/30

(52) U.S. Cl. 435/52; 435/41; 435/441; 435/471; 435/69.1; 435/89; 536/23.1

(57) ABSTRACT

The present invention relates to a method for preparation of a modified host cell which comprises the steps of (a) transfecting a host cell with at least one compound of interest to which a label is covalently coupled and (b) isolating the transfected host cell, wherein the label provides to the host cell a non-inheritable trait. Modified host cells according to the invention can be directly separated from the non-modified host cell. To this end use is made of labels, which can be monitored at the modified cells (such as fluorescent labels) and which enable separation of the modified and non-modified host cells by suitable means. In case of fluorescent labels use can be made of a Fluorescent Activated Cell Sorter. Suitable compounds of interest according to this invention are compounds, which enable to change permanently or transiently a metabolic property of the host cell. Examples of compounds are polynucleotides, proteins or metabolites. The host cells modified according to the present invention can be used for the production of proteins, metabolites and cell biomass.

The present invention relates to a method for preparing a modified host cell. It also relates to a method for the production of proteins, metabolites and cell biomass making use of said modified host cells.

Genetic modification of cells can be done either by classical strain improvement or by metabolic pathway engineering (MPE; Khetan and Hu (1999) In: Manual of Industrial Microbiology Biotechnology (Eds. Demain and Davies) 717-724). Classical strain improvement has been proven to be a powerful technology for introduction of (random) mutations and the subsequent selection of strains with new features (e.g. improved metabolite production, altered morphology or growth on a specific substrate). However, some mutations such as new functionalities, new pathways and selection markers cannot be introduced by classical mutagenesis. In order to attain these latter mutations, one has to apply MPE, relying on molecular biology, in particular the introduction of new genetic material into the host. In literature, many examples can be found illustrating successful applications of MPE (Crawford et al. (1995) Biotechnology 13:58-62; Khetan et al. (1996) Ann. N Y Acad. Sci. 782:17-24.). Moreover, using modern MPE technology it is possible to introduce several new features in one round (Ye et al. (2000) Science 287:303-305). In all these cases new and/or additional genetic material has been introduced into the host strain.

The introduction of DNA into a cell can be done using a wide range of known technologies (e.g. electroporation, conjugation, particle bombardment, injection, etc). It depends on the organism used, which technology will be applicable.

The genes can be introduced on an artificial carrier, like plasmids, cosmids or chromosomes, or, alternatively, integrated into the host genome. Alternatively, non-inheritable features, like anti-sense oligonucleotides, short-hairpin RNA's, non-coding RNA's, proteins or metabolites, can be introduced in the host cell to trigger permanent or temporary modifications in the metabolism.

Afterwards, only the cells that have taken up the particular molecules need to be isolated.

In literature, almost exclusively DNA and RNA molecules are used to modify the metabolism of a cell, but in most cases these do not encode a protein with a selectable feature, like resistance against a certain metabolite. And as most cells do not take up the molecules there is a need for an efficient selection procedure to select the recombinant cells.

Moreover, if episomal constructs or non-inheritable features are used, a constant selection pressure must be applied once the modified cells are selected. Otherwise, these features are gradually being lost from the dividing microorganism. This is done with the aid of so-called selection markers. This can be a gene that encodes a protein, which will give resistance to a certain metabolite (like an antibiotic or a high metal concentration) or will enable the cells to grow on a specific carbon source.

Even though these procedures are elegant, selective and widely applied, there are certain drawbacks, especially for industrial applications. In most industrial processes it is not possible to apply a constant selection pressure using antibiotics.

Lacking the possibility of constant selection pressure by antibiotics, the use of episomal constructs is only applicable in a few industrial processes, wherein the growth conditions (mostly the composition of the media in the case of micro-organisms) will be the selective pressure. An example is the use of auxotrophic growth markers. To this end an organism first must be mutated to select strains with the necessary growth deficiencies. In the second round these growth deficiencies can be used as a selective pressure. An intact copy of the mutated feature is co-introduced with the heterologous genes on the same plasmid. When the right growth conditions are applied the cells can only grow when they contain this plasmid with the functional genes. This will put strict limitations on how a production process is run and on the variation in media optimization. This will in turn limit the applicability of this technology. Most industrial organisms do not contain any auxotrophic marker and are often polyploids (for example see: Hadfield et al. (1995) Curr. Genet. 27:217-228), which makes it difficult to select auxotrophic derivatives. Therefore, dominant markers as hygromycin resistance are used to select transformants of industrial strains. This renders the newly derived strain with a piece of heterozygous DNA that limits new transformation rounds. Moreover, in plant species it has been shown that these elements might cause instabilities (for example see: Windels et al. (2001) Eur. Food Res. Technol. 213:107-112).

Also, there is a limited set of selection markers available for each organism (see for example: Van den Berg and Steensma (1997) Yeast 13:551-559), which will hamper the possibilities of this approach.

More widely used in industrial strains is the stable integration of new DNA in the genome. In most cases this is done by co-integration of the selection marker (U.S. Pat. No. 6,051,431). In the first screen one selects the strains that contain the selection marker, and in the second screen one selects the strains also containing the gene(s) of interest. The major drawback is that the marker is also stably integrated in the genome, and the same marker cannot be used for a second transformation round. Two further drawbacks are that there is a need for two successive selection/screening rounds and that more DNA then necessary is being introduced. This additional DNA-load can be a burden for the cell (see for example: Elmore et al. (2001) Agronomy J. 93:408412). But more important, the ongoing public debate on correlation between the use of antibiotic resistance markers in industrial processes and the increase in natural antibiotic resistance makes alternatives essential. Apart from the examples mentioned above, there are many alternative tools available in which the selection marker can be removed from the genome after stable integration of the gene(s) of interest (see for examples: Lyznik et al. (1995) Plant J. 8:177-186; Selten et al. (1997) U.S. Pat. No. 6,051,431; Yang and Hughes (2001) Biotechniques 31:1036-1041). However these alternatives are time consuming and can cause additional instabilities to the cell-line of interest. Moreover, in most cases (for example see: Lyznik et al. (1995)) there is still some DNA left on the chromosome.

As the optimization of (industrial) organisms is highly dependent on recombinant DNA techniques for introduction of new and/or more efficient metabolic pathways to the host strain, there is a need for a new technology. Ideally, this new technology should be applicable to all organisms, independent of the actual DNA sequences used, not dependent on antibiotic or other selection markers, yielding stable modified cell lines and repeatable useable to previously transformed cell lines. None of the technologies available today can for fill all of these demands. Currently, selection of new strains with DNA integrated into the host genome, always involves the co-integration of a selectable marker, which limits the rate of new transformation rounds using these derivative strains. Using standard available labeling techniques in a different application, the present invention surprisingly solves this problem. Using non-inheritable markers as fluorescein molecules covalently bound to the gene of interest or non-gene encoding markers as Green Fluorescent Protein mixed with the gene of interest, cells, which have taken up these markers can be separated from the marker-free cells and subsequently be inoculated in fresh medium. During cell division the transformants derived in this way take up standard nutrients from the medium to synthesize new DNA strands and other cell constituents. During this process they will loose the artificial non-inheritable marker(s) as these are not present in or can be synthesized from standard culture media. Therefore, the cell line becomes marker-free as a rule. Subsequently, the integrated gene(s) of interest will result in the altered capabilities of the production organism. So, the method described by this invention also surprisingly demonstrates that the temporal covalent attachment of fluorescein molecules to a gene of interest does not change the properties and/or the function of the gene of interest. Furthermore, the transformants can directly be used for new transformation rounds.

According to one embodiment the method for preparation of a modified host cell according to the present invention comprises the steps of (a) transfecting a host cell with at least one compound of interest to which a label is covalently coupled and (b) isolating the transfected host cell, wherein the label provides to the host cell a non-inheritable trait.

According to a further embodiment the present invention relates to a method for preparation of a modified host cell comprising the steps of (a) transfecting a host cell with at least one compound of interest to which a label is covalently coupled and (b) isolating the transfected host cell, wherein the label provides to the host cell a non-inheritable trait and wherein isolation of the transfected host is established by direct separation of the host cells containing said label from host cells not containing said label.

According to a further embodiment the present invention relates to a method for preparing a modified host cell comprising the steps of (a) transfecting a host cell with at least one compound of interest covalently coupled to a label which provides to the host cell a non-inheritable trait, and (b) isolating the transfected host cell containing the label as obtained in step a) by using means that can distinguish and separate said transfected host cell containing said label from non-transfected host cell.

Subsequent to said separation step the transfected host cell can be cultured in order to multiply the organisms.

With the “compound of interest” is meant according to the present invention any compound, which enables to change permanently, or transiently a metabolic property of the host cell. Examples of “compounds of interest” are polynucleotides (for example nucleotide fragments such as a gene, a promoter, an expression cassette, a terminator, a plasmid, a small oligonucleotide which is able to interfere with a mRNA, RNA, hexose nucleic acid (HNA), peptide nucleic acid (PNA), or locked nucleic acid (LNA), antisense oligonucleotide, short hairpin RNA, non-coding DNA), proteins or metabolites.

The modified host cell can be used to produce oligonucleotides (for example RNA), (recombinant) proteins (for example antibodies, proteases, lipases, chymosin), primary or secondary metabolites (for example anti-infectives such as P-lactam antibiotics and building blocks, amino acids, and clavulanic acid), or biomass (such as yeast cells) on a laboratory or an industrial scale, for screening or commercial purposes, respectively.

With “modified host cell” according to the present invention is meant a host cell, which is permanently or transiently changed in its composition of DNA, RNA, proteins and/or metabolites. Such change might be established by over-expression of one or more gene(s); or by suppression of the expression of one or more gene(s); or by knocking out one or more gene(s); or by altering the regulation of one or more gene(s); or by gene silencing of one or more gene(s); or by so-called RNA interference, whereby the messenger RNA levels of one or more gene(s) are decreased or abolished; or by enzymes that trigger a response or divert cellular metabolism; or by metabolites that trigger a response or divert cellular metabolism. Preferably, the modified host according to the present invention expresses an additional gene (or genes) as compared to the unmodified host; or in the modified host a gene (or genes) is inactivated by deletion or interruption of said gene(s); or by binding of a complementary nucleotide sequence to said gene (or genes) or to part of said gene (or genes); or a combination of these methods. In the modified host according to the present invention DNA expression levels and/or RNA expression levels and/or protein expression levels and/or metabolite levels may be temporarily or structurally altered.

“Transfecting a host” comprises transferring the at least one compound of interest, and optionally also other elements to provide stability to the host cell, or a secondary compound of interest, or an element for integration into the host cell.

The “label” according to the present invention comprises any label that is directly detectable or that can be made detectable. Preferably, the label is a fluorescent label, a luminescent label, a chemo luminescent label, an enzymatic label, a magnetic label, an antigenic label or a radioactive label. Suitable fluorescent labels are rhodamine, fluorescein, alexa fluor, cascade blue, tetramethylrhodamine, and Texas red. Suitable enzymatic labels include alkaline phosphatase, luciferase and β-galactosidase. Suitable luminescent labels include acridinium esters, luminol, isoluminol, oxalate esters, dioxetanes, and luciferin. Suitable chemo luminescent labels include reactive aminoallyl-modified dNTP's, nitro blue tetrazolium, lucigenin. Suitable radioactive labels include ³H, ³²P, ¹⁵N, ¹³C, and ¹⁴C. Suitable magnetic labels include magnetic beads containing iron, nickel or cobalt. Suitable antigenic labels include FITC, digoxin and biotin. For proper detection these antigenic labels can be reacted with conjugated antibodies (such as anti-digoxin), or with conjugated streptavidine.

A “non-inheritable trait” (as opposed to an inheritable trait) means a structural or functional characteristic, which upon multiplication of the host will not be multiplied itself and subsequently will be diluted and lost in the progeny of the host. A suitable host cell according to the present invention is a prokaryotic organism, such as bacteria, or a eukaryotic organism such as yeast, fungi, plant cells or animal cells.

With “means to monitor and separate the transfected host” is meant any equipment or treatment, which may enable detection of the label covalently bound to the transfecting compound on single cell level and which converts the detected label to a signal that can be monitored. Subsequently, the means to monitor the label can also separate the single cell that contains the label from cells that do not contain said label. In case the label is fluorescent or luminescent, using any apparatus that monitors electromagnetic radiation, such as infrared or ultraviolet light, X-rays, microwaves, and visible light may detect these labels. In case the label is a fluorescent label, the means to monitor said label may be a fluorescence detection apparatus. Preferably, it may be a Fluorescent Activated Cell Sorter (FACS). In case the label is a radioactive label, the means to monitor said label may be a radiation detection apparatus such as a Geiger Muller teller or ¹³C-NMR with cell sorting function. In case the label is a (chemo) luminescent label, the means to monitor said label may be a FACS adapted with an additional luminometer. In case the label is enzymatic, the means to monitor said label may be a FACS. In case the label is magnetic, the means to monitor said label may be an autoMACS™.

The present invention also relates to a method for the preparation of a desired compound by a transformed host cell comprising the steps of a) transfecting a host with at least one polynucleotide involved in the production of said desired compound and which is covalently coupled to a label which provides to the host cell a non-inheritable trait, b) isolating the transfected host, c) culturing the transfected host under proliferating conditions, d) culturing the transfected host under conditions wherein the desired compound is produced and e) isolating the desired compound from the culture broth.

In a preferred embodiment the polynucleotide modifies the titer, stability, isolation and/or activity of said desired compound. Preferably the desired compound so produced is a protein, more preferably an enzyme.

The present invention also relates to a method for the preparation of a desired metabolite by a transformed host cell comprising the steps of a) transfecting a host cell with at least one polynucleotide involved in the production of said desired metabolite and which is covalently coupled to a label which provides to the host cell a non-inheritable trait, b) isolating the transfected host cell, c) culturing the transfected host cell under proliferating conditions, d) culturing the transfected host cell under conditions wherein the desired metabolite is produced, and e) isolating the desired metabolite from the culture broth. In a preferred embodiment of this latter method the polynucleotide is selected from the group consisting of DNA, RNA, short hairpin RNA, non-coding RNA, LNA, HNA and PNA. In a further embodiment of this method the polynucleotide modifies the cellular metabolism via redirecting metabolic fluxes towards said metabolite. Preferably, the desired metabolite is a primary metabolite such as an amino acid, a steroid or a nucleotide. More preferably, the desired metabolite is a secondary metabolite, such as an antibiotic, a vitamin, an anti-infective, a macrolide, a polyketide, a pheromone, an alkaloid or a drug.

The present invention also relates to a method for the preparation of a desired biomass from a transformed host cell comprising the steps of a) transfecting a host cell with at least one polynucleotide involved in the production of said desired biomass and which is covalently coupled to a label which provides to the host cell a non-inheritable trait, b) isolating the transfected host, c) culturing the transfected host under proliferating conditions, d) culturing the transfected host under conditions wherein the desired biomass is produced, and e) isolating the desired biomass. Advantageously, the desired biomass is a yeast cell. In another embodiment the desired biomass is a biocatalyst. In a further embodiment the desired biomass comprises screenable cells for drug discovery.

### DESCRIPTION OF THE FIGURES

FIG. 1 Cells of Penicillium chrysogenum transfected with purified enhanced Green Fluorescent Protein (A: Light microscopy; B: Fluorescence microscopy with excitation at 460 nm and emission at 525 nm).

FIG. 2 Cells of Penicillium chrysogenum transfected with fluorescent oligonucleotides (A: Light microscopy; B: Fluorescence microscopy with excitation at 460 nm and emission at 525 nm).

FIG. 3 Cells of Penicillium chrysogenum transfected with fluorescent Dextran 10.000 MW. A combination of light and fluorescence microscopy with excitation at 460 nm and emission at 525 nm was used.

FIG. 4 Fluorescence activated cell sorter profiles from Penicillium chrysogenum protoplasts with excitation at 460 nm and emission at 525 nm (A: Non-transfected protoplasts; B: Protoplasts stained with vacuolar marker MFY-64 (Molecular Probes); C: Protoplasts transfected with unlabeled pGBDEL4L; D: Protoplasts transfected with fluorescent labeled pGBDEL4L).

FIG. 5 Cells of Penicillium chrysogenum transfected with fluorescent-labeled plasmid pGBDEL4L (A: Light microscopy; B Fluorescence microscopy with excitation at 460 nm and emission at 525 nm).

FIG. 6 Data generated by the FACS after sorting the control transformations, on the left side the scatter plot and on the right side the fluorescence plot. Background emission was eliminated; the square in the corner of the fluorescence plot is the background emission boundary.

FIG. 7 Data generated by the FACS from the pRS4255 transformation, on the left side the scatter plot and on the right side the fluorescence plot. The square in the corner of the fluorescence plot is the background emission boundary as described for the control transformations. Dots outside this corner represent cells that have fluorescence higher then the background.

### EXAMPLES

### Example 1

### Transfection of Cells with Green Fluorescent Protein

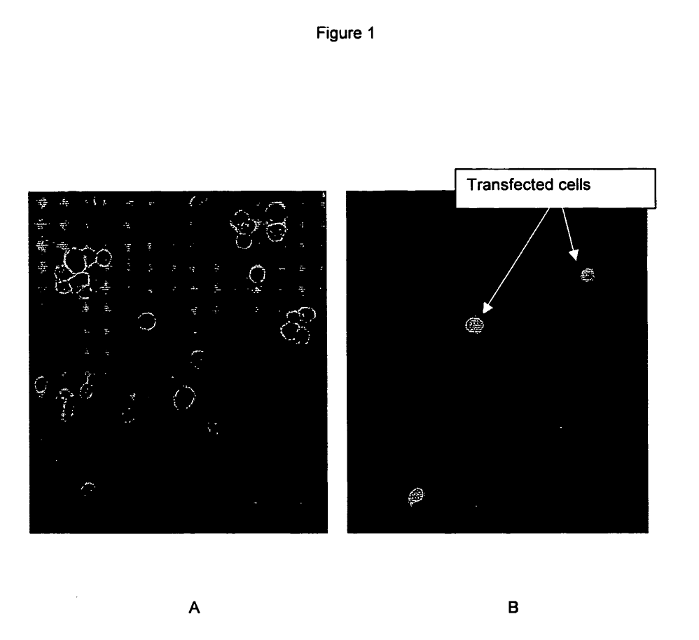

Penicillium chrysogenum was cultivated for 48 hours in standard glucose medium. Cells were washed and cell walls were degraded using 4 mg/ml Novozym (NOVO/Nordisk). The obtained protoplasts were washed twice and used for transfection as described by Theilgaard et al. (2001, Biotechnol. Bioeng. 72:379-388). During transfection 20 nM of purified, recombinant Enhanced Green Fluorescent Protein (Clontech; cat# 8365-1) was added. After transfection protoplasts were washed twice with SCT (Sorbitol, 1.2 M; CaCl₂, 50 mM; Tris-HCl, 10 mM; pH 7.5). Several competent cells were shown to have taken up the fluorescent protein (see FIG. 1).

This example demonstrates that applying directly detectable signals (in this case fluorescence) covalently linked to a protein as a means of selecting cells results in desired cells in which that protein could trigger a (transient) metabolic change.

### Example 2

### Transfection of Cells with Fluorescent Labeled DNA Oligonucleotides

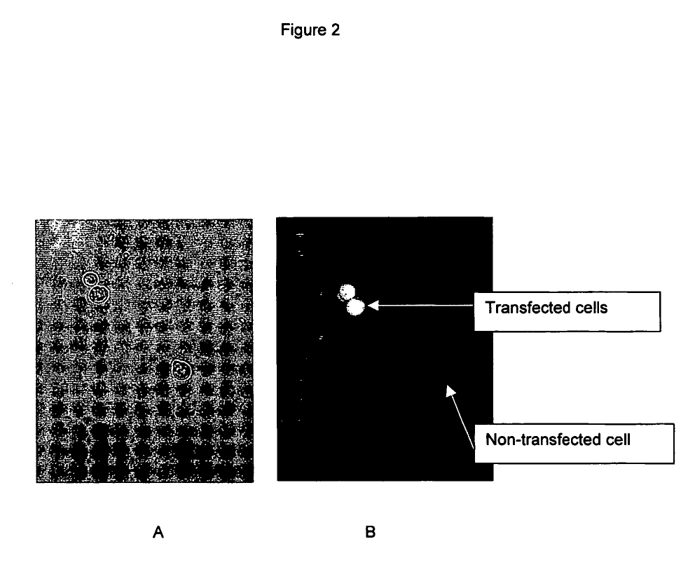

Competent Penicillium chrysogenum protoplasts were obtained and processed as described in example 1. Two complementary, fluorescent-labeled oligonucleotides were used: fluorescein-5′-GGGGAAATGGACAAAA-3′ and fluorescein-5′-TTTTGTCCATTTCCCC-3′ (Life Technologies). Respectively 38 and 48 nmol of both oligonucleotides were added to the transfection mixture (according to Theilgaard et al. (2001)), resulting in a final concentration of 46 μM during the actual transfection. After transfection several fluorescent cells could be visualized (see FIG. 2).

This example demonstrates that applying directly detectable signals (in this case fluorescein) covalently coupled to oligonucleotides as a means of selecting desired cells results in cells in which those oligonucleotides can trigger (transient) metabolic changes.

### Example 3

### Transfection of Cells with Fluorescent Labeled Dextran

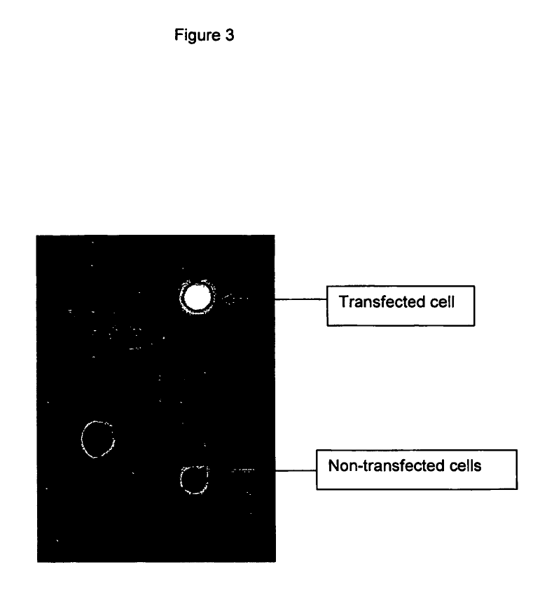

Competent Penicillium chrysogenum protoplasts were obtained and processed as described in example 1. A fluorescent labeled hydrophilic polysaccharide, Dextran 10.000 MW (Molecular Probes, cat# D-7169) was used in a final concentration of 50 μM during the actual transfection. After transfection several fluorescent cells could be visualized (see FIG. 3).

This example demonstrates that applying directly detectable signals (in this case fluorescence) covalently coupled to a compound as a means of selecting desired cells results in cells in which those compounds could trigger (transient) metabolic changes.

### Example 4

### Transfection of Cells with Fluorescent Labeled DNA

Transfection

Competent Penicillium chrysogenum protoplasts were obtained and processed as described in example 1. Five μg of plasmid pGBDEL4L, containing an expression cassette to drive expression of the enzyme acetamidase in the fungus (Selten et al. (1999) U.S. Pat. No. 5,876,988), was labeled using the Label IT Nucleic acid labeling reagent (Mirus; cat# MIR3200). The label, 5-carboxy fluorescein, was attached to the guanine residues of the plasmid according to the supplier's manual. The sample was purified using the purification column. The efficiency of labeling was checked on agarose gels. One μg of fluorescent-labeled plasmid was used for transfection. Cells were washed once with SCT and used directly after for selection, firstly on fluorescence and secondly on agar plates containing acetamide as the sole nitrogen source.

Selection for Fluorescent Cells Using a Fluorescence Activated Cell Sorter (FACS)

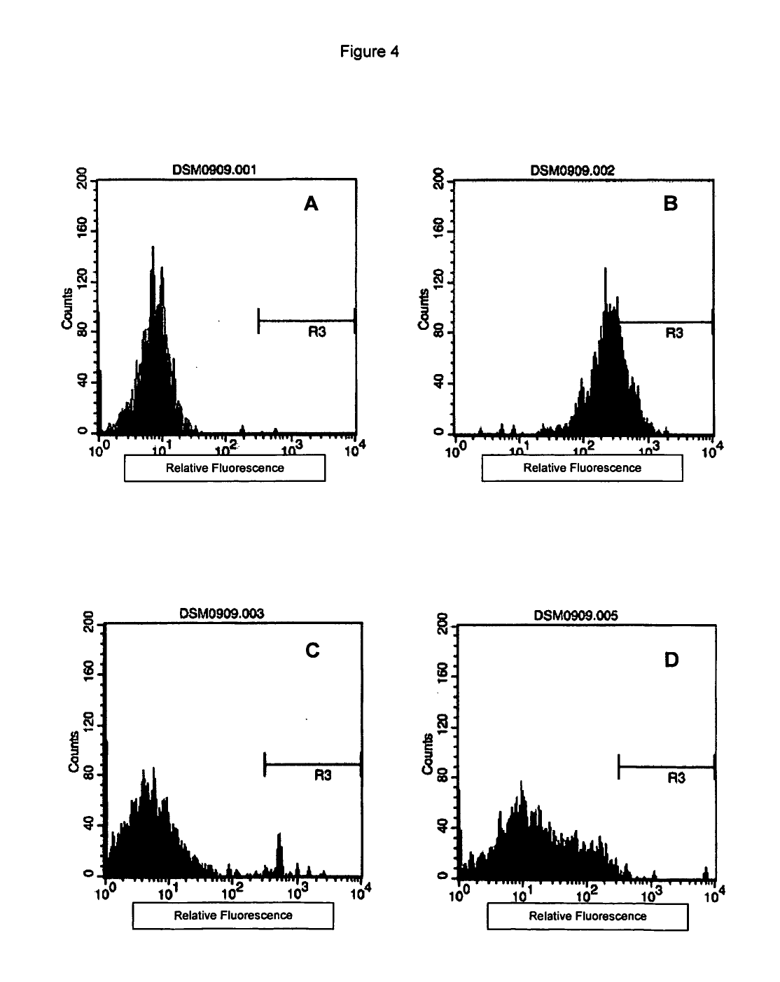

A FACS was used for isolation of the transfected cells (as described by De Nooij-Van Dalen et al. (2001) Genes Chromosomes Cancer 30:323-335). After transformation approximately 10⁶ cells were re-suspended in 4 ml of STC. Sorted cells were collected in STC with 5% BSA (w/v). As controls non-transfected protoplasts (FIG. 4A), protoplasts stained with the fluorescent vacuolar marker MDY-64 (FIG. 4B) and protoplasts transfected with unlabelled pGBDEL4L (FIG. 4C) were sorted. It was possible to obtain at least a 100-fold difference in labeled and unlabeled protoplasts (compare FIGS. 4A and 4B). Therefore, we concluded that using this method we could isolate transfected cells with a fluorescent DNA molecule (FIGS. 4C and 4D). Using unlabeled pGBDEL4L most cells show the same fluorescence as non-transfected protoplast. There is a small peak visible at a higher relative fluorescence (around 6-7×10²), but this was due to the clumping of some cells after the polyethylene glycol-mediated transfection. This level was set as a threshold value for the selection of fluorescent cells in the experiment with labeled pGBDEL4L. We were able to select approximately 3200 cells that showed a relative high fluorescence (around 6-7×10³; see FIG. 4D).

Fluorescence of Selected Cells

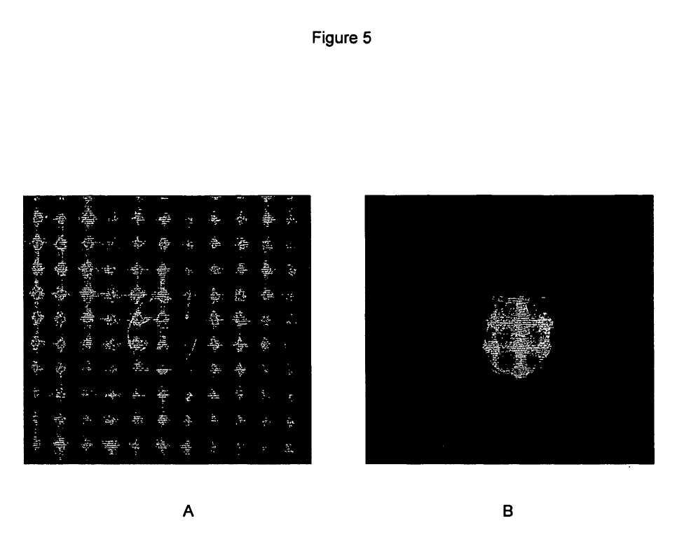

A sample of the 3200 cells selected was used for fluorescence microscopy. FIG. 5 shows that a bright fluorescence is visible within these cells.

Selection for Presence of Functional amdS Gene

Another sample of the 3200 cells selected were plated on media with acetamide as the sole nitrogen source. After a week several colonies were obtained on these media. Showing that the label did not affect the functionality of the amdS gene and that we were able to select stable transformants solely on the presence of fluorescence. So, these cells lost the non-inheritable fluorescein marker, but retained the gene of interest.

This example demonstrates that applying directly detectable signals (in this case fluorescein) covalently coupled to DNA as a means of selecting and sorting the desired, modified cells results in cells in which the polynucleotide of interest triggers permanent metabolic changes.

### Example 5

### Integration of Fluorescent DNA in Penicillium cells

Logarithmic growing Penicillium chrysogenum cells were transformed using the protocol described in example 1. Linear DNA (0.75 μg amdS fragment obtainable after digesting pGBDEL4L of example 4 with the restriction enzymes EcoRI and SspI) was labeled using the Label IT® Nucleic Acid Labeling kit MIR3200 from MIRUS. The labeling reaction was carried out using 35 μl milliQ, 5 μl 10*Mirus labeling A, 5 μl DNA (˜0.15 μg/μL), 5 μl Label IT reagent. After 3 hours incubation at 37° C., the DNA was purified by ethanol precipitation. The labeling was checked by agarose electrophoresis on an agarose gel without ethidium bromide. The labeled DNA was made visible by UV-light. As control Penicillium cells transformed with water were used. Cells were analyzed using a FACS Vantage (BD Biosciences). Cells transformed with non-labeled DNA were used for determining the background emission. After adjusting the FACS for background, the Penicillium cells transformed with fluorescent labeled DNA were sorted.

The results shown in table 1 demonstrate that protoplasts can resist the pressure in the FACS. Due to some clumping of protoplasts high and low scatter populations were isolated (see table 1). Only, cells with high scatter gave amdS positive clones (see sample E, table 1), demonstrating integration of fluorescent labeled DNA. So, after growing on synthetic media these cells lost the non-inheritable fluorescein marker, but retained the gene of interest.

TABLE 1
Results of FACS sorting of Penicillium transformants, the
CFU's counted on selective and non-selective media.

| | | | Volume | | | |
|---|---|---|---|---|---|---|
| | | | plated | | | |
| # | Sample description | FACS sorting | out | Media | CFU | CFU/ml |
| A | Before FACS sorting | None | 200 μl | amdS-regeneration | 60 | 300 |
| B | Before FACS sorting | None | 250 μl | amdS-regeneration | 53 | 200 |
| C | Sorted high scatter | high scatter high | 5 μl | non-selective | 13 | 2600 |
| | | fluorescence | | regeneration | | |
| D | Sorted high scatter | high scatter high | 200 μl | non-selective | >300 | >1500 |
| | | fluorescence | | regeneration | | |
| E | Sorted high scatter | high scatter high | 100 μl | amdS-regeneration | 2 | 20 |
| | | fluorescence | | | | |
| F | Sorted low scatter | low scatter high | 100 μl | non-selective | 7 | 70 |
| | | fluorescence | | regeneration | | |

### Example 6

### Transformation and Selection of Yeast Cells

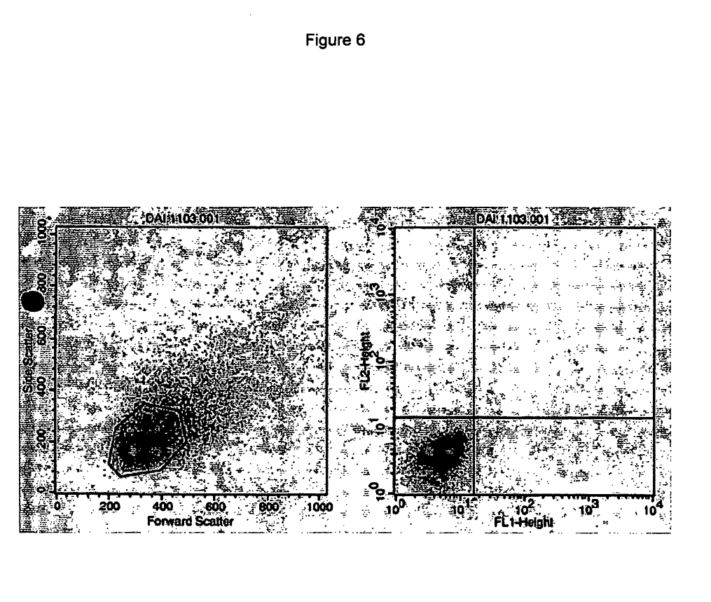

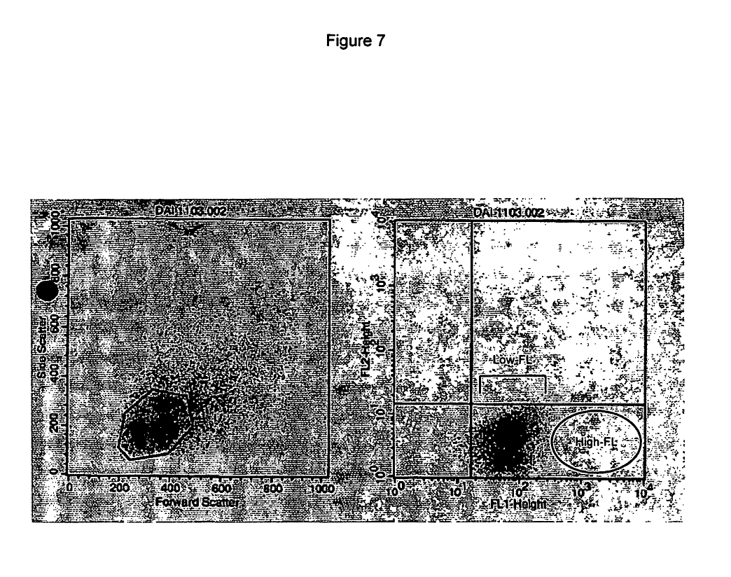

Logarithmic growing yeast cells of strain CEN PK 2-1 c were transformed using the standard LithiumAcetate protocol. Two μg pRS425 (Christianson and Sikorski (1992) Gene 110:119-122) was labeled using the Label IT® Nucleic Acid Labeling kit MIR3200 from MIRUS. The labeling reaction was carried out using 35 μl milliQ, 5 μl 10*Mirus labeling A, 5 μl DNA (˜0.4 μg/μL) and 5 μl Label IT reagent. After 3 hours incubation at 37° C., the DNA was purified by ethanol precipitation. The labeling was checked by agarose electrophoresis on an agarose gel without ethidium bromide. The labeled DNA was made visible by UV-light. As control yeast cells transformed with water were used. Cells were analyzed using a FACS Vantage (BD Biosciences). FIG. 6 shows a typical plot representing of water-transformed cells; FIG. 7 shows a typical plot representing of pRS425-transformed cells.

Most of the pRS425-transformed cells are more fluorescent then the background. From these cells several extreme fluorescent cells could be sorted (High-FL fraction indicated by the circle in FIG. 7); extreme fluorescent cells were spotted directly on a YEPD plate. Although fluorescent, cells displaying low signals (the Low-FL fraction in FIG. 7) were no real transformants indicated by plating them out on selective YNB medium (see samples 4 and 5, table 2). Only cells from the High-FL fractions were shown to be real transformants after testing the colonies appearing on non-selective YEPD plates (see sample 7, table 2) via re-streaking on selective YNB plates. So, after growing on synthetic media these cells lost the non-inheritable fluorescein marker but retained the gene of interest.

TABLE 2
Results of FACS sorting of yeast transformants, the CFU's
are counted on selective and non-selective media.

| | | | Volume plated | | | |
|---|---|---|---|---|---|---|
| # | Sample description | FACS sorting | out | Media | CFU | CFU/ml |
| 1 | Before FACS sorting pRS425 | none | 200 μl | YNB | 123 | 600 |
| 2 | After FACS sorting pRS425 | low fluorescence | 100 μl | YEPD | >500 | >5000 |
| 3 | After FACS sorting pRS425 | low fluorescence | 10 μl | YNB | 0 | — |
| 4 | After FACS sorting pRS425 | low fluorescence | 100 μl | YNB | 0 | — |
| 5 | After FACS sorting pRS425 | low fluorescence | 10 μl | YEPD | 200 | 20000 |
| 6 | After FACS sorting pRS425 | low fluorescence | 100 μl | YEPD | >500 | >5000 |
| 7 | After FACS sorting pRS425 | high fluorescence | directly 50 cells | YEPD | 12 | — |

1. A method for preparation of a modified host cell comprising:
a) transfecting a host cell with at least one compound of interest to which a label is covalently coupled and
b) isolating the transfected host cell;
wherein the label provides to the host cell a non-inheritable trait.

2. A method according to claim 1, wherein isolation of the transfected host cell is established by direct separation of the host cells containing said label from host cells not containing said label.

3. A method according to claim 1, wherein isolation of the transfected host cell is established by using means that can distinguish and separate said transfected host cell containing said label from non-transfected host cells.

4. A method according to claim 1, wherein the label is selected from the group consisting of a fluorescent label, a luminescent label, a chemoluminescent label, a magnetic label, an antigenic label, an enzymatic label, and a radioactive label.

5. A method according to claim 3, wherein the label is a fluorescent label and the means for detection is a Fluorescent Activated Cell Sorter (FACS).

6. A method according to claim 1, wherein the transfected host cell of step b) is subsequently cultured.

7. A method according to claim 1, wherein the compound of interest is a compound able to change permanently or a transiently metabolic property of the host cell.

8. A method according to claim 1, wherein the compound of interest is selected from the group consisting of polynucleotides, proteins, and metabolites.

9. A method according to claim 1, wherein the modified host cell is a prokaryotic cell, a eukaryotic cell, a mammalian cell or a plant cell.

10. A method for preparation of a desired compound by a transformed host cell comprising:
a) transfecting a host with at least one polynucleotide involved in production of said desired compound and which is covalently coupled to a label which provides to the host cell a non-inheritable trait,
b) isolating the transfected host,
c) culturing the transfected host under proliferating conditions,
d) culturing the transfected host under conditions wherein the desired compound is produced, and
e) isolating the desired compound from the culture broth.

11. A method according to claim 10, wherein the polynucleotide is selected from the group consisting of DNA, RNA, short hairpin RNA, non-coding RNA, LNA, HNA, and PNA.

12. A method according to claim 10, wherein the polynucleotide modifies the titer, stability, isolation and/or activity of said desired compound.

13. A method according to claim 10, wherein the desired compound is a protein.

14. A method according to claim 10, wherein the desired compound is an enzyme.

15. A method for preparation of a desired metabolite by a transformed host cell comprising:
a) transfecting a host cell with at least one polynucleotide involved in production of said desired metabolite and which is covalently coupled to a label which provides to the host cell a non-inheritable trait,
b) isolating the transfected host cell,
c) culturing the transfected host cell under proliferating conditions,
d) culturing the transfected host cell under conditions wherein the desired metabolite is produced, and
e) isolating the desired metabolite from the culture broth.

16. A method according to claim 15, wherein the polynucleotide is selected from the group consisting of DNA, RNA, short hairpin RNA, non-coding RNA, LNA, HNA, and PNA.

17. A method according to claim 15, wherein the desired metabolite is a primary metabolite.

18. A method according to claim 15, wherein the desired metabolite is an amino acid, a steroid or a nucleotide.

19. A method according to claim 15, wherein the desired metabolite is a secondary metabolite.

20. A method according to claim 19, wherein the desired secondary metabolite is an antibiotic, a vitamin, an anti-infective, a macrolide, a polyketide, a pheromone, an alkaloid or a drug.

21. A method for preparation of a desired biomass by a transformed host cell comprising:
a) transfecting a host cell with at least one polynucleotide involved in production of said desired biomass and which is covalently coupled to a label which provides to the host cell a non-inheritable trait,
b) isolating the transfected host,
c) culturing the transfected host under proliferating conditions,
d) culturing the transfected host under conditions wherein the desired biomass is produced, and
e) isolating the desired biomass.

22. A method according to claim 21, wherein the polynucleotide is selected from the group consisting of DNA, RNA, short hairpin RNA, non-coding RNA, LNA, HNA, and PNA.

23. A method according to claim 21, wherein the desired biomass is a yeast cell.

24. A method according to claim 21, wherein the desired biomass comprises a biocatalyst.

25. A method according to claim 21, wherein the desired biomass comprises screenable cells for drug discovery.

26. A polynucleotide for use in a method according to claim 15, which modifies the cellular metabolism via redirecting metabolic fluxes towards said metabolite.


SEQUENCE LISTING (2 sequences)

SEQ ID NO: 1 - DNA, 16 bp, Artificial Sequence
  Description of artificial sequence synthetic primer
ggggaaatggacaaaa

SEQ ID NO: 2 - DNA, 16 bp, Artificial Sequence
  Description of artificial sequence synthetic primer
ttttgtccatttcccc